(19) United States
(12) Patent Application Publication
(10) Pub. No.: US 2011/0282245 A1
(43) Pub. Date: Nov. 17, 2011

(54) SYSTEM FOR ANALYZING ABNORMAL HIP JOINT CONDITIONS

(75) Inventors: Michael Decker, Vail, CO (US); Kevin Shelburne, Golden, CO (US)

(73) Assignee: STEADMAN-PHILIPPON RESEARCH FOUNDATION, Vail, CO (US)

(21) Appl. No.: 12/942,140

(22) Filed: Nov. 9, 2010

Related U.S. Application Data
(60) Provisional application No. 61/259,877, filed on Nov. 10, 2009.

(51) Int. Cl. A61B 5/103

(52) U.S. Cl. 600/587

(57) ABSTRACT

A device for assessing hip joint conditions comprises a support structure including a first platform area and a second platform area spaced from each other. The device includes a first footrest rotationally coupled to the first platform, a second footrest rotationally coupled to the second platform, a first recording device coupled to the first footrest and adapted to record the angular movement of the first footrest, and a second recording device coupled to the second footrest and adapted to record the angular movement of the second footrest.

### PRIORITY AND CROSS REFERENCE TO RELATED APPLICATIONS

This application claims the benefit of Provisional U.S. Patent Application No. 61/259,877 filed on Nov. 10, 2009. The details of Application No. 61/259,877 are incorporated by reference into the present application in their entirety and for all proper purposes.

### FIELD OF THE INVENTION

The present invention relates to a system and method for diagnosing and analyzing abnormal hip joint conditions.

### BACKGROUND OF THE INVENTION

Femuro acetabular impingement (FAI) is a common cause of hip pain.^{(1, 2)} Recently, FAI has been recognized as a cause of labral tears, which may lead to early arthritis of the hip.^{(3, 4)} FAI is particularly common in the athletic population, with an incidence of hip injuries ranging from 3.1% to 8.4% in this subset of patients.^{(5, 6)} Clinically, patients with FAI refer groin pain, especially during activities with flexion and internal rotation, or extension and external rotation. On physical examination, patients present with positive anterior impingement test and FABER test.⁽²⁾ Radiographic examination is of paramount importance for the evaluation of FAI, as bony abnormalities are closely related to labral tears.⁽⁷⁾ Anteroposterior pelvic and cross-table lateral views are done in order to access the bony anatomy, and to classify FAI into one group: CAM, pincer or mixed.⁽⁸⁾ The alpha angle is measured in the cross-table lateral view or in MRI tilted axial cuts in order to evaluate the head-neck junction as described by Notzli⁽⁹⁾, values greater than 42° are suggestive of a head-neck offset deformity.⁽⁸⁾ After FAI is diagnosed, patients are put in a rehabilitation program. If after six weeks, conservative treatment fails; surgery is indicated. FAI can be addressed either by open or arthroscopic techniques. The goal of the surgery is to address the bony abnormality, reducing the acetabular retroversion and restoring head-neck offset, the labral pathology is treated too, with better results achieved after labral refixation versus labral debridement.^{(10, 11)} Recent studies with long term follow-up have shown consistent results with hip arthroscopy.⁽¹²⁾ Furthermore, when FAI, labral and cartilage pathology are treated earlier, better outcomes are reported, especially regarding conversion to joint replacement and earlier return to sports.^{(13, 14)}

Because FAI can lead to severe consequences and as earlier diagnosis leads to better results, it is appropriate to screen patients with bony abnormality of the hip joint. Screening is of special interest in athletic populations involved in sports of high risk of labral tears, such as hockey and golf. After screening, patients could have different training programs, where their hip would be put in less stress, and hopefully they would have a smaller probability of developing labral tears. Meyer et al. have performed a screening program in college football players with promising results.⁽¹⁵⁾ One possible way of screening would be taking X-rays and looking directly to bony abnormality, however this may constitute a too invasive screening method.

The diagnosis and treatment of hip pathology spanning conservative and surgical measures is increasing. For instance, there is an increasing awareness of the diagnosis of femoroacetabular impingement (FAI) particularly in young individuals 16 to 40 years old. Symptoms may include restriction of movement, ‘clicking’ of the hip joint, and pain. These symptoms can occur during prolonged sitting, walking and sports participation. Several anatomic characteristics in combination or in isolation may result in FAI. The typical features include increased anterior coverage of the femoral head from acetabular retroversion, a non-spherical femoral head, short anterior head-neck offset, retroversion of the femoral head, and a deep acetabulum. These anatomical characteristics constrain hip motion by causing an abnormal mechanical contact between the femur and acetabulum leading to labral detachment, chondral lesions and osteoarthritis. The aim of arthroscopic FAI surgery is to decrease hip pain and improve hip range of motion (ROM) by alleviating the femoral abutment against the acetabular rim. Although there is evidence to support increased ROM after FAI surgery, most clinical outcome studies for FAI are criticized because there is a lack of quantitative data, particularly during dynamic motions.

The most common clinical assessment of dynamic hip function is the deep squat. The patient is required to have the feet point directly forward and bend at the hips, knees and ankles such that the motions of these joints track directly over their feet. An abnormal mechanical relationship between the femur and acetabulum will prevent normal downward motion and reduce the depth of the squat. However, FAI patients can perform a deep squat to the same depth as individuals with mechanically normal hips if they alter the mechanical relationship between the femur and acetabulum by changing how much the hips abduct or adduct and externally or internally rotate. The accurate measurement of this dynamic compensatory motion may 1) quantify the severity (magnitude) of mechanical impingement; 2) assess the efficacy of surgical intervention for FAI; and 3) serve as a screening tool to identify potential ‘at risk’ individuals or athletes.

Although present devices are functional, they are not sufficiently accurate or otherwise satisfactory. Accordingly, a system and method are needed to address the shortfalls of present technology and to provide other new and innovative features.

### SUMMARY OF THE INVENTION

In accordance with one aspect, a hip deck assessment device has been developed with the goal of screening patients with bony abnormality of the hip in a non invasive method. In a broad implementation, the hip deck is a fixed support deck with two rotating platforms mounted on the top surface of the support deck. In use, a patient stands on the deck, with each foot on one of the rotating platforms. Then, the patient performs a squat. During the squatting motion, his feet are free to rotate on each of the platforms and this rotation can be easily measured with a scale integrated into the fixed deck.

In accordance with another aspect, the hip deck includes a measurement device that provides a rapid and accurate clinical assessment of hip motion during a deep squat. In addition, the device measures the weight placed on each foot, rotational force and the depth of the squat. The hip deck device can also be used to measure hip or knee rotation in several other patient populations performing many different functional activities during standing and while seated.

In accordance with another aspect, a device for assessing hip joint conditions comprises a support structure including a first platform area and a second platform area spaced from each other. The device also includes a first footrest rotationally coupled to the first platform, a second footrest rotationally coupled to the second platform, a first recording device coupled to the first footrest and adapted to record the angular movement of the first footrest, and a second recording device coupled to the second footrest and adapted to record the angular movement of the second footrest.

Exemplary embodiments of the present invention that are shown in the drawings are summarized below. These and other embodiments are more fully described in the Detailed Description section. It is to be understood, however, that there is no intention to limit the invention to the forms described in this Summary of the Invention or in the Detailed Description. One skilled in the art can recognize that there are numerous modifications, equivalents and alternative constructions that fall within the spirit and scope of the invention as expressed in the claims.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawings, wherein:

FIG. 1 is an overview drawing of a hip deck device as used by a patient;

FIG. 2 is a close up drawing of one of the platform areas that supports a patient's foot;

FIG. 3 shows one embodiment of a hip deck device constructed in accordance with aspects of the present invention;

FIG. 4 is a partially exploded view of a hip deck device constructed in accordance with aspects of the present invention; and

FIG. 5 is an embodiment of an electronic sensor and recording device used in connection with one aspect of the present invention.

### DETAILED DESCRIPTION

In a broad sense, a hip deck device constructed in accordance with aspects of the present invention consists of two rotational footrests each connected to a pedestal that is capable of supporting the weight of a patient. Each rotational footrest can either be locked into place and prevented from rotating or allowed to freely rotate 360°. Two connecting rods link the pedestals of each rotational footrest. The side-to-side distance between the rotational footrests can be adjusted by sliding a pedestal along the connecting rods to be either closer or further away from the other pedestal.

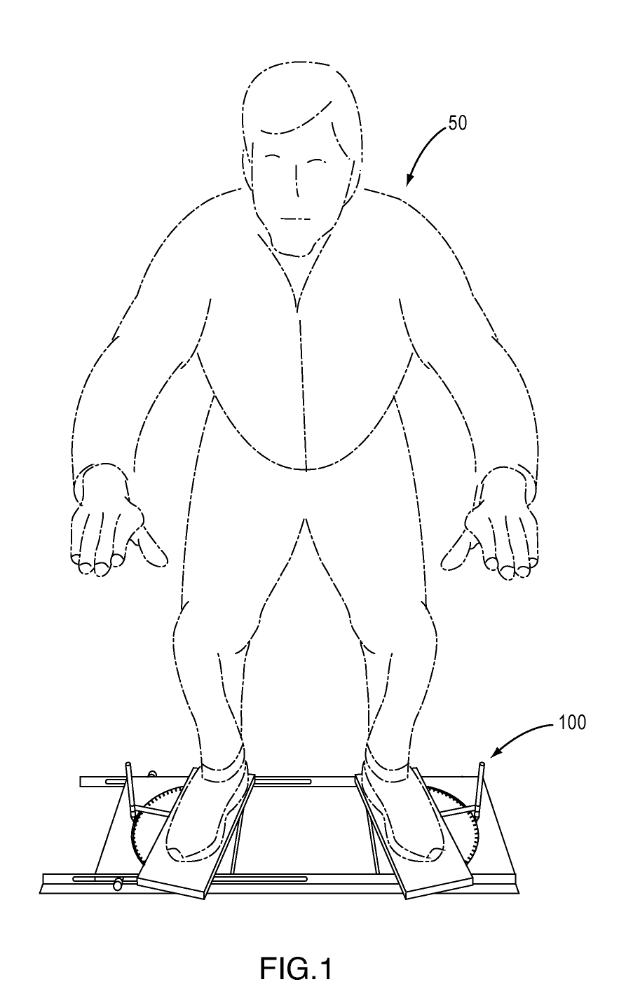

FIG. 1 shows a hip deck device 100 constructed in accordance with aspects of the present invention as used in one example to measure hip rotation while a patient 50 performs a squat or other motion that involves engagement of the hip physiology. The device 100 is placed on the floor, and the patient 50 stands on the hip deck with each foot on a rotational footrest 120 and 140 separated by a pre-determined distance and mounted on a pair of support beams 102 and 104. The footrests 120 and 140 are rotationally mounted on corresponding platforms 110 and 115. Each of the rotational footrests have the ability to lock in place to prevent unintended movement when the patient steps on the platforms 110 and 115. When the rotational footrests 120 and 140 are unlocked and free to rotate, the patient performs a squat and the feet are allowed to rotate in a horizontal plane parallel to the floor. FIG. 1 illustrates a patient in the squatting position with the corresponding rotation of the footrests 120 and 140. The platforms 110 and 115 may in one embodiment be adapted to slide along the support beams so that the distance between the footrests 120 and 140 can be adjusted for different patients.

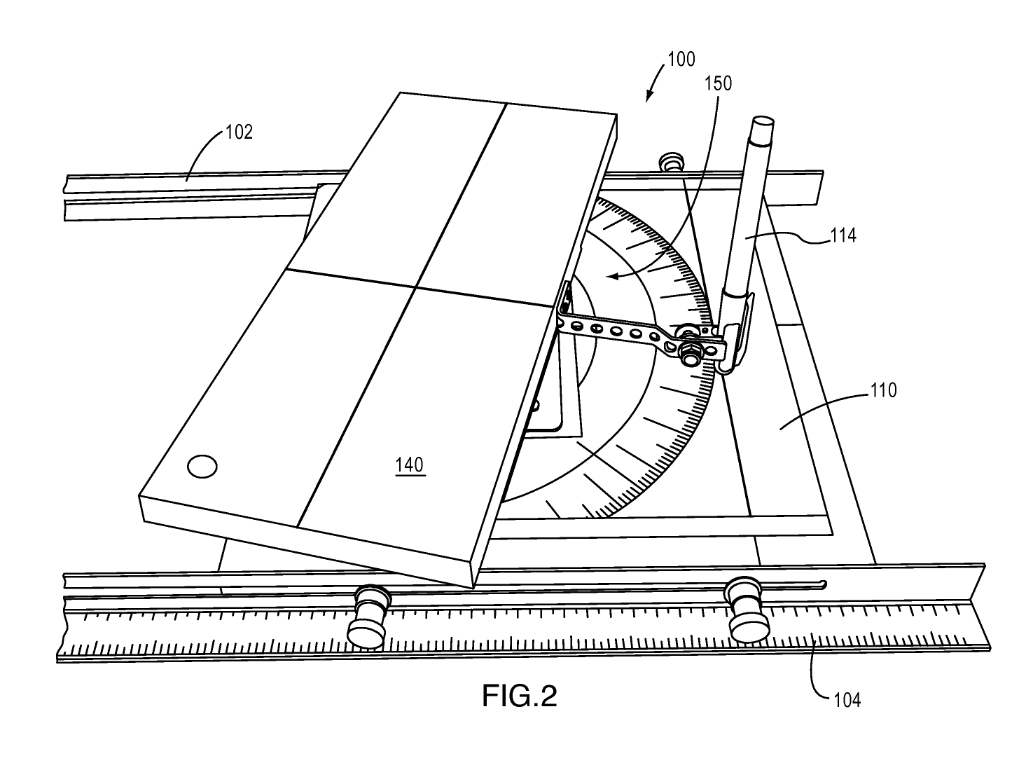

FIG. 2 shows a close up view of one of the support platforms 110, the footrest 140 and a measuring and recording system 150 that is used to track and record the angle of rotation that occurs during the squatting motion performed by the patient. In one embodiment, a simple pen and paper is used to record the motion and provide quantitative measurement of the rotational position of the footrests and therefore the patient's feet. Each rotational footrest 120 and 140 is equipped with a recording device and in other embodiments, mechanical sensors are incorporated to permanently record data such as rotational force, vertical force and rotational position associated with the particular patient. Any of these sensors may be integrated into a computerized recording and monitoring system to provide additional data recording and analysis capabilities.

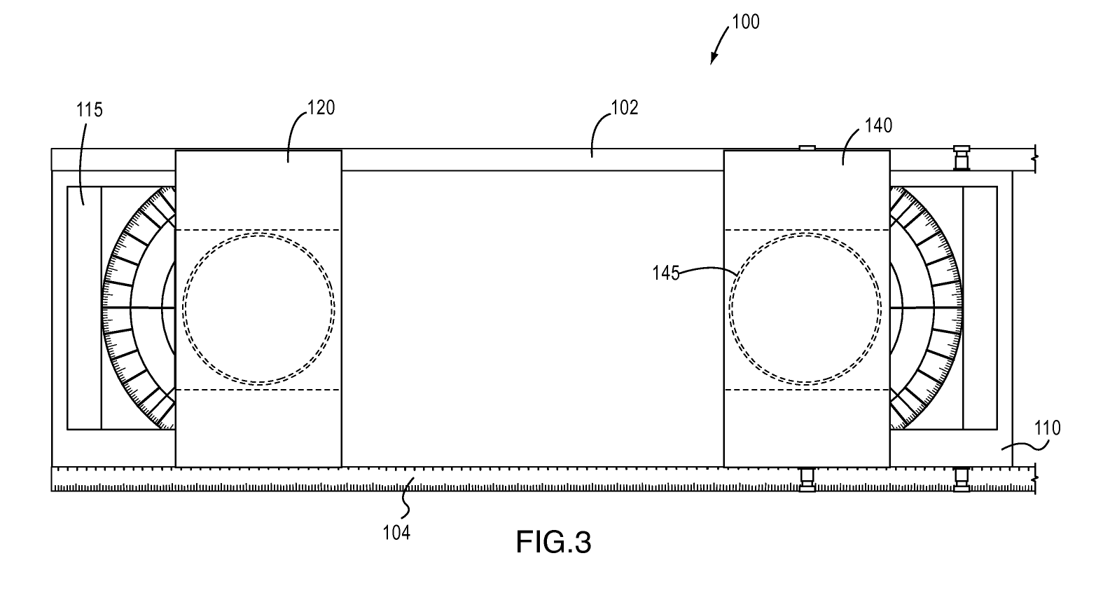

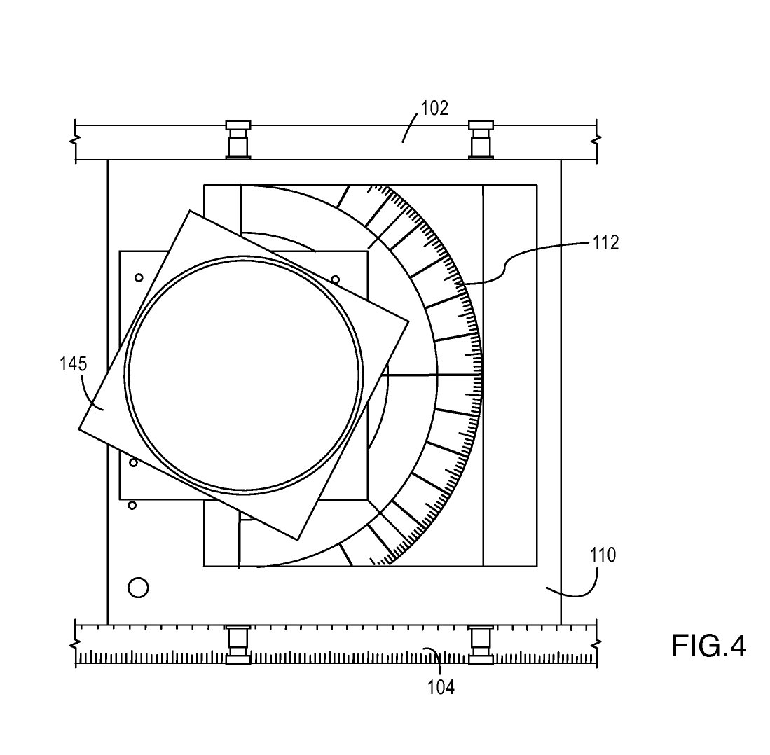

FIG. 3 is an isolated view of the hip deck device 100 showing the features described above in conjunction with FIG. 2. FIG. 4 is an exploded view of the hip deck device 100. In FIG. 4, one embodiment of the rotational platform 140 is shown with the rotational element 145 positioned beneath the platform 140, thus allowing it to rotate freely under the movement of a patient. For illustration purposes, a pen 114 or other recording device in combination with a protractor 112 is shown as one way to record to rotational position of the platform 140.

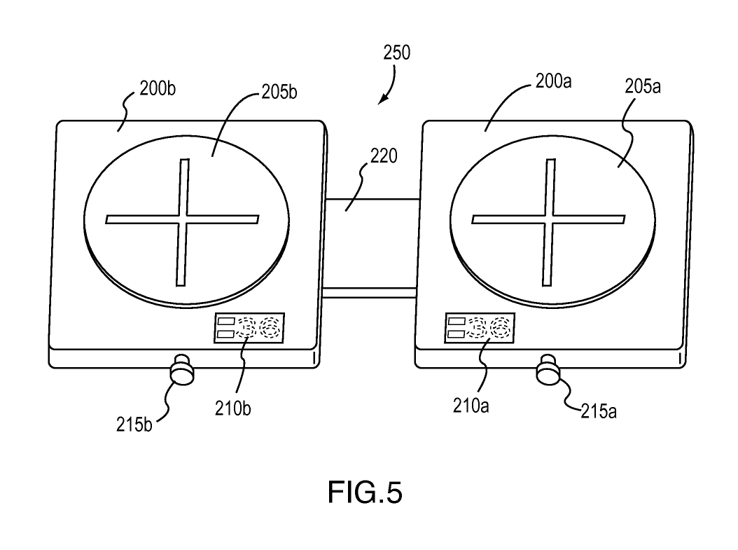

FIG. 5 illustrates one embodiment of a rotational sensor system 250 that can be used in place of the pen/protractor embodiment discussed above. The sensor system 250 includes a pair of sensor housings 200a and 200b integrated with rotational elements 205a and 205b. In addition to sensing and recording the angular rotation of the rotational elements 205a and 205b, sensor housings 200a and 200b are in some embodiments adapted to record one or more of weight, strain, force and other measurements associated with the patient 50. Digital displays 210a and 210b display the measurements taken by the sensor system 250 and also relay that information to a computer system or database for further processing. Locking devices 215a and 215b are included to secure the rotational elements 205a and 205b in place. The distance between sensors housings 200a and 200b can be slidably adjusted via a plate 220 incorporated into the hip deck device 100.

In accordance with various other embodiments it is contemplated that one or more of the following data outputs can be captured during an analysis session using a hip deck device constructed in accordance with aspects of the present invention: a) starting hip internal/external rotation position, b) maximum hip internal/external rotation, c) hip internal/external rotation ROM, d) weight placed on each foot during activity, e) hip, knee, ankle and back flexion angles, f) pelvic pitch (anterior/posterior pelvic tilt), and g) squat depth.

Other advantages associated with using a hip deck device constructed in accordance with aspects of the present invention include its ease of use, it is light and portable for easy transport, it is simple to set up and put away in a clinic, it can be adjusted for different patients and activities, and it is designed to provide a permanent record of output.

Depending on the exercise performed, a hip deck constructed in accordance with aspects of the present invention can be integrated with other recording devices and/or designed with different sensors. For example, mechanical measurements recorded with a pen and protractor may be replaced by electronic sensors in the platform that transcribe rotational position and force, weight and squat depth. In addition, these measurements may be recorded using a hand held display or with an interfaced laptop or desktop computer. Mechanical measurements recorded with pen and protractor or force sensors may also be replaced by sensors located on the foot and lower limb that record the rotation. These can include optical methods such as a camera and reflective marker set-up, or electromagnetic and gyroscopic sensor arrays to provide additional or alternative rotational measurements of the pelvis, hip, knee and ankle as well as squat depth.

Those skilled in the art can readily recognize that numerous variations and substitutions may be made in the invention, its use and its configuration to achieve substantially the same results as achieved by the embodiments described herein. Accordingly, there is no intention to limit the invention to the disclosed exemplary forms. Many variations, modifications and alternative constructions fall within the scope and spirit of the disclosed invention as expressed in the claims.

### REFERENCES

1. McCarthy J C, Lee J A. Arthroscopic intervention in early hip disease. Clin Orthop Relat Res. 2004 Dec. 1(429):157-62.
2. Philippon M J, Maxwell R B, Johnston T L, et al. Clinical presentation of femoroacetabular impingement. Knee Surg Sports Traumatol Arthrosc. 2007 Aug. 1; 15(8):1041-7.
3. Ganz R, Parvizi J, Beck M, et al. Femoroacetabular impingement: a cause for osteoarthritis of the hip. Clin Orthop Relat Res. 2003 Dec. 1(417):112-20.
4. McCarthy J C, Noble P C, Schuck M R, et al. The Otto E. Aufranc Award: The role of labral lesions to development of early degenerative hip disease. Clin Orthop Relat Res. 2001 Dec. 1(393):25-37.
5. Feeley B, Powell J, Muller M, et al. Hip Injuries and Labral Tears in the National Football League. Am J Sports Med. 2008 Sep. 18; 36(11):2187-95.
6. Borowski L, Yard E, Fields S, et al. The Epidemiology of US High School Basketball Injuries, 2005-2007. Am J Sports Med. 2008 Aug. 25; 36(12):2328-35.
7. Wenger D E, Kendell K R, Miner M R, et al. Acetabular labral tears rarely occur in the absence of bony abnormalities. Clin Orthop Relat Res. 2004 Sep. 1(426):145-50.
8. Clohisy J, Carlisle J, Beaule P, et al. A Systematic Approach to the Plain Radiographic Evaluation of the Young Adult Hip. J Bone Joint Surg Am. 2008 Nov. 1; 90(Supplement 4):47-66.
9. Nötzli H P, Wyss T F, Stoecklin C H, et al. The contour of the femoral head-neck junction as a predictor for the risk of anterior impingement. J Bone Joint Surg Br. 2002 May 1; 84(4):556-60.
10. Espinosa N, Rothenfluh D A, Beck M, et al. Treatment of femoro-acetabular impingement: preliminary results of labral refixation. J Bone Joint Surg Am. 2006 May 1; 88(5):925-35.
11. Philippon M J, Briggs K K, Yen Y M, et al. Outcomes following hip arthroscopy for femoroacetabular impingement with associated chondrolabral dysfunction: minimum two-year follow-up. J Bone Joint Surg Br. 2009 Jan. 1; 91(1):16-23.
12. Byrd J, Jones K. Prospective Analysis of Hip Arthroscopy with 10-year Followup. Clin Orthop Relat Res. 2010 Mar. 21; 468(3):741-6.
13. Philippon M, Weiss D, Kuppersmith D, et al. Arthroscopic Labral Repair and Treatment of Femoroacetabular Impingement in Professional Hockey Players. Am J Sports Med. 2010 Jan. 1; 38(1):99-104.
14. Beck M, Leunig M, Parvizi J, et al. Anterior femoroacetabular impingement: part II. Midterm results of surgical treatment. Clin Orthop Relat Res. 2004 Jan. 1(418):67-73.
15. Meyer J L, Philippon M, Briggs K, et al. Preparticipation Hip Evaluation of College Football Players to Identify Athletes at Increased Risk of Chondral Labral Dysfunction of the Hip. ISAKOS; Japan 2009.

What is claimed is:

1. A device for assessing hip joint conditions, comprising:
a support structure, the support structure including a first platform area and a second platform area spaced from each other on the support structure;
a first footrest rotationally coupled to the first platform;
a second footrest rotationally coupled to the second platform;
a first recording device coupled to the first footrest and adapted to record the angular movement of the first footrest; and
a second recording device coupled to the second footrest and adapted to record the angular movement of the second footrest.

2. The device for assessing hip joint conditions of claim 1, wherein the first platform area and the second platform area are integrated into a single structure.

3. The device for assessing hip joint conditions of claim 1, wherein the first platform area and the second platform area each include a protractor.

4. The device for assessing hip joint conditions of claim 1, further including a first mechanical device that couples the first footrest to the first platform and allows the first footrest to rotate on the first platform area.

5. The device for assessing hip joint conditions of claim 4, further including a second mechanical device that couples the second footrest to the second platform and allows the second footrest to rotate on the second platform area.

6. The device for assessing hip joint conditions of claim 1, wherein the support structure comprises a pair of substantially parallel beams that secure the first and second platform areas.

7. The device for assessing hip joint conditions of claim 1, wherein each of the first and second footrests include a molded area to receive a patient's foot.

8. The device for assessing hip joint conditions of claim 1, wherein at least one of the first and second recording devices is a writing instrument.

9. The device for assessing hip joint conditions of claim 1, wherein at least one of the first and second recording devices is a electronic sensor for measuring angular displacement.

10. The device for assessing hip joint conditions of claim 1, further comprising a device for measuring the weight of the patient.

11. The device for assessing hip joint conditions of claim 1, further comprising a device for measuring the angular force exerted by a patient.

12. The device for assessing hip joint conditions of claim 1, wherein at least one of the first and second recording devices is a camera.

13. The device for assessing hip joint conditions of claim 1, wherein at least one of the first and second recording devices is a reflective marker.

14. The device for assessing hip joint conditions of claim 1, wherein at least one of the first and second recording devices is a an electromagnetic sensor array.

15. The device for assessing hip joint conditions of claim 1, wherein at least one of the first and second recording devices is a gyroscopic sensor array.

16. The device for assessing hip joint conditions of claim 1, wherein the device is adapted to record one or more of the rotational measurements of the pelvis, hip, knee and ankle.

17. The device for assessing hip joint conditions of claim 1, wherein the device is adapted to record squat depth.

18. The device for assessing hip joint conditions of claim 1, further comprising a hand held display for viewing and recording the angular movement of at least the first footrest.

19. The device for assessing hip joint conditions of claim 6, wherein the first and second platform areas are slidably movable along the first and second beams.

20. A device for assessing hip joint conditions, comprising:
a support structure,
a first footrest rotationally coupled to the support structure;
a second footrest rotationally coupled to the support structure;
a first recording device coupled to the first footrest and adapted to record the angular movement of the first footrest; and
a second recording device coupled to the second footrest and adapted to record the angular movement of the second footrest.
